SEQUENCE LISTING (26 sequences)

SEQ ID NO: 1 - DNA, 21 bp, Artificial
  primer
gcttgcaaagtgacggtcatt

SEQ ID NO: 2 - DNA, 21 bp, Artificial
  primer
ctatccaaatgcagtgaactc

SEQ ID NO: 3 - protein, 11 aa, Influenza virus
YGRKKRRQRRR

SEQ ID NO: 4 - protein, 20 aa, Artificial
  HA2 Domain
GLFGAIAGFIENGWEGMIDG

SEQ ID NO: 5 - protein, 20 aa, Artificial
  HA2 domain
GLFEAIAEFIEGGWEGLIEG

SEQ ID NO: 6 - protein, 20 aa, Artificial
  diINF-7 domain
GLFEAIEGFIENGWEGMIDG

SEQ ID NO: 7 - protein, 12 aa, Hepatitis B virus
LLNQLAGRMIPK

SEQ ID NO: 8 - protein, 12 aa, Hepatitis B virus
TLDHVLDHVQTM

SEQ ID NO: 9 - protein, 14 aa, Human papillomavirus
SYFILRRRRKRFPY

SEQ ID NO: 10 - protein, 15 aa, Human papillomavirus
SYYMLRKRRRKRLPY

SEQ ID NO: 11 - protein, 14 aa, Human papillomavirus
LYYFIRKKRKRVPY

SEQ ID NO: 12 - protein, 24 aa, Artificial
  Histatin 5 domain
DSHAKRHHGYKRKFHEKHHSHRGY

SEQ ID NO: 13 - protein, 14 aa, Artificial
  dhvar4
KRLFKKLLFSLRKY

SEQ ID NO: 14 - protein, 14 aa, Artificial
  dhvar4 domain
LLLFLLKKRKKRKY

SEQ ID NO: 15 - protein, 16 aa, Drosophila sp.
RQIKIWFQNRRMKWKK

SEQ ID NO: 16 - protein, 7 aa, Artificial
  poly-Arg
RRRRRRR

SEQ ID NO: 17 - protein, 12 aa, Artificial
  PTD-5
RRQRRTSKLMKR

SEQ ID NO: 18 - protein, 27 aa, Artificial
  synthetic construct
GWTLNSAGYLLGKINLKALAALAKKIL

SEQ ID NO: 19 - protein, 16 aa, Drosophila sp.
PQIKIWFQNRRMKWKK

SEQ ID NO: 20 - protein, 21 aa, Artificial
  Buforin II
TRSSRAGLQFPVGRVHRLLRK

SEQ ID NO: 21 - protein, 10 aa, Mouse
VPMLKPMLKE

SEQ ID NO: 22 - protein, 18 aa, Rat
RGGRLSYSTTTFSTSTGR

SEQ ID NO: 23 - protein, 21 aa, Mouse
KETWWETWWTEWSQPKKKRKV

SEQ ID NO: 24 - protein, 15 aa, Saccharomyces cerevisiae
SDLWEMMMVSLACQY

SEQ ID NO: 25 - protein, 12 aa, Mouse
TSPLNIHNGQKL

SEQ ID NO: 26 - protein, 9 aa, Influenza virus
RKKRRQRRR

(19) United States
(12) Patent Application Publication
(10) Pub. No.: US 2009/0325866 A1
(43) Pub. Date: Dec. 31, 2009

(54) NON-ACTIVATED WNT INHIBITION POLYPEPTIDES AND METHOD FOR PREPARING THE SAME

(76) Inventors: Jung Moon Kim, Seoul (KR); Jung Kook Kim, Seoul (KR); Tae Han Kim, Gyeonggi-do (KR); Jong Suk Lee, Seoul (KR); Jong In Yook, Seoul (KR)

Correspondence Address: MOORE & VAN ALLEN PLLC, P.O. BOX 13706, Research Triangle Park, NC, 27709, US

(21) Appl. No.: 12/090,787

(22) PCT Filed: Nov. 30, 2006
(86) PCT No.: PCT/KR2006/005134

§ 371 Date: Jun. 26, 2008

(30) Foreign Application Priority Data
Nov. 30, 2005 (KR) 10-2005-0115281

(51) Int. Cl. A61K 38/17; C07K 14/47; C12N 15/63; C12N 1/21; C12P 21/02

(52) U.S. Cl. 514/12; 530/350; 435/320.1; 435/252.3; 435/69.7

(57) ABSTRACT

The present invention relates to non-activated Wnt inhibition polypeptides (WIPs) containing: (a) a protein transduction domain (PTD) which enables said WIPs to permeate a cell membrane without the aid of a cell membrane receptor; and (b) a Wnt antagonist domain which is inactive by itself, but is activated in mammalian cells and then secreted out of the cells to function to inhibit Wnt signal transduction. Also, the invention relates to a method for preparing said non-activated WIPs, and a pharmaceutical composition containing said WIPs as active ingredients. Said non-activated WIPs can be produced in large quantities through the culture of bacteria such as E coli., and are biochemically inactive before being administered into the human body, and thus the production cost thereof is only one several tenths of that of previously known active proteins (sFRPs, DKKs, etc.) having uses similar thereto, and the isolation/purification and handling/administration processes thereof are significantly simple and convenient. When said non-activated WIPs are administered in vivo, they will have the effects of inhibiting the invasive growth and metastasis of cancer cells and treating immune diseases, such as rheumatoid arthritis by pharmacological mechanisms different from those of the previously known sFRPs or DKKs.

### TECHNICAL FIELD

The present invention relates to non-activated Wnt inhibition polypeptides (WIPs) containing: (a) a protein transduction domain (PTD) which enables said WIPs to permeate a cell membrane without cell membrane receptor; and (b) a Wnt antagonist domain (WAD) which is inactive by itself, but is activated in mammalian cells, and then secreted out of the cells to function to inhibit Wnt signaling, a method for preparing said non-activated WIPs, and a pharmaceutical composition containing said WIPs as an active ingredient.

### BACKGROUND ART

The Wnt gene was derived from the fact that a gene (wingless) regulating the development of Drosophila is the same as the oncogene of mice (Int-1) (Cell, 50:649, 1987). Since then, it is known that the Wnt signaling system consists of a group of about 20 genes, and the 20 genes known to transduce signals into cells through specific receptors, i.e., Frizzled receptors (Fz) and LRP5/6 co-receptors. The signals transduced though Fz and LRP are passed through Dvl (Dishevelled), APC (adenomatous polyposis coli) and Axin and ultimately regulate the activation of β-catenin by GSK3-β (glycogen synthase kinase 3-β), thus, resulting in that β-catenin is a key regulator of the Wnt signaling system (Science, 303:1483, 2004; Genes Dev., 11:3286, 1997). Meanwhile, β-catenin binds to TCF (T-cell factor)/Lef (lymphoid enhancing factor) to regulate the transcription of various genes, and the abnormality of Wnt signaling components, including β-catenin, are the most typical oncogenic factor (Biochem. Biophy. Acta, 1653:1, 2003). Two features of human malignant tumors, which are different from normal cells or tissues, are continued cell division, and metastasis to other organs. Particularly, s the metastasis of epithelium-derived tumors (95% of malignant tumors) to other sites plays a decisive role against the survival of patients. However, the cellular mechanism of cancer cell metastasis is not well-known. Till now, the intercellular protein of epithelial cells, E-cadherin, has been thought to be an important factor of regulating the metastasis, but the mechanism of E-cadherin expression regulation of cancer cells is not well known. It was recently reported that a human Snail gene directly inhibits the mRNA transcription of E-cadherin (Nature Cell Biol., 2:84, 2000; Nature Cell Biol., 2:76, 2000; Nature Rev. Mol. Cell Biol., 3:155, 2002), and more recently, it is known that the Wnt signaling system inhibits GSK3-β to regulate the phosphorylation and half-life of not only β-catenin, but also Snail (J. Biol. Chem., 280:11740, 2005). This indicates that the Wnt signaling system regulates not only oncogenesis, but also the metastasis of cancer cells. Ultimately, this suggests that, if the Wnt signaling system is blocked, the growth and metastasis of cancer cells can be inhibited.

Meanwhile, the most typical pathogenic mechanism of rheumatic arthritis (RA) is the proliferation of activated FLS (fibroblast-like synoviocytes) leading to the degenerative change of joints, and it was recently reported that the proliferation of FLS is induced by Wnt, and the use of a Wnt antagonist strongly inhibits the activation of FLS, thus the report suggests that the Wnt antagonist can be used as a new therapeutic method of RA (Rhuematol., 44:708, 2005). In addition, in fibrotic diseases such as pulmonary fibrosis, the activation of Wnt/β-catenin signal transduction is known to cause pulmonary fibrosis resulting in breathing difficulty (Am. J. Pathol., 162:1495, 2003), and thus the Wnt signaling system is understood to be not only the mechanism of stem cell differentiation (Nature, 432:324, 2004), but also the pathogenic mechanism of various diseases. Therefore, it is believed that, if a method capable of effectively interrupting the Wnt signaling system is suggested, epoch-making methods for treating various diseases, including cancer, can be developed.

In higher animals, including particularly human beings, a group of proteins capable of inhibiting Wnt signaling is present. These proteins are secreted, like the Wnt protein, and bind to the Fz receptor or the LRP co-receptor, thus acting as antagonists of the Wnt signaling (J. Cell Sci., 116:2627, 2003). These Wnt lo antagonists can be classified into the following two categories: (1) an sFRP (secreted Frizzled-related protein) group, including sFRP1-5, sizzled 1-2, Crescent, WIF-1 and Coco (J. Cell Sci., 116:2627, 2003); and (2) a Dickkopf (DKK) group, including DKK1-4 and Soggy (Nature, 411:255, 2001).

Accordingly, if these Wnt antagonists promote the phosphorylation of the Snail gene, and thus increase the transcription of E-cadherin to inhibit the invasive growth and metastasis of epithelium-derived cancer cells (J. Biol. Chem., 280:11740, 2005), an epoch-making therapeutic method of inhibiting the metastasis of cancer cells can be suggested, and also effective therapeutic methods against various diseases induced by Wnt signaling can be provided.

But, methods for effectively delivering protein to cells and tissues cause various technical problems. Recently as protein transduction domain (PTD; Joliot, A. et al., Nature Cell Biol., 6:198, 2004; US patent 2006/0222657A1), which can effectively deliver proteins into cells, was found, it has been possible to deliver various peptides, which can result in biological activity, into cells.

TAT (transactivator of transcription), AntHD (Drosophila homeoprotein atennapedia transcription protein), VP22 (virus protein22) peptide and mph-1-btm (mouse transcription inhibitory factor-1-biomolecule transduction mortif), Penetratin, BuforinII, Transportan, Ku70, Prion, pVEC, Pep-1, PTD-5, KALA (Joliot, A. et al., Nature Cell Biol., 6:198, 2004; Kabouridis, P. S., Trends Biotechnol., 21:498, 2003) is known as PTD. The PTDs are typically cationic in nature. The PTDs are transported into cytoplasm by entering lipid raft in an endosome without cell membrane receptor to dissociate a carrier which is in the form of endosome in cells.

In the case of most widely known TAT, it was found that the part, which consists of 9 basic amino acid (RKKRRQRRR) existing in TAT protein, plays an important role in passing through a cell membrane. However, there are problems in that TAT (RKKRRQRRR) has low transmission rates due to large amount of amino acids having a positive charge and TAT-fusion protein has less effective stability and duration in cells than those in nature, since it is activated by refolding of a protein in a cell after being delivered into the cell in the denatured state, not in the native state (Schwartz, S. R. et al., Trends in Cell Biology, 10:290, 2000), and TAT-fusion protein is not effectively delivered to cytoplasm, nucleus or organelle, since it remains bound to a carrier in cells after being introduced through endocytosis. In the case of AntHD (Drosophila homeoprotein atennapedia transcription protein), it has a problem in that it can be fused with only proteins having the length less than 100 amino acids.

Haemagglutinin2 is known as another substance that can effectively deliver proteins into cells (HA2 domain; Skehel, J. J. et al., Biochem. Society Med., 10:310, 25 2004; Jehangir, S. W. et al., Nature Medicine, 10:310, 2004; Vaccaro, L. et al., Biophy. J., 88:25, 2005). It is capable of avoiding a decrease in the biological activity by macropinosome or endosome and is an influenza membrane fusion protein capable of increasing cell transmission rate.

Haemagglutinin (HA) of influenza is a kind of glycoprotein which is a component of viral envelope, and it is involved in mediating by having virus attached to a target cell or by fusing target cell membrane with viral envelope membrane. In the case of general viral infection, virus bound to the cell surface is introduced into an endosome to be exposed to relatively low pH. The pH change triggers conformational changes. The conformational changes lead to increased exposure of the amino terminus of HA as well as fusion between the viral envelope and the endosomal membrane.

HA is composed of two polypeptide segments, HA1 and HA2. HA1 segment forms sialic acid-binding site to attach it to the host cell surface. The HA2 segment forms a membrane-spanning anchor, and the amino-terminal site acts in fusion reaction mechanism.

Since haemagglutinin2 (HA2 domain) does not have B-cell recognition site, although it has more than one T-helper cell recognition site, it doesn't induce antibody response to itself while it causes T-dependent immune response to antigen combined with HA2 domain. Because HA2 segment contains hydrophobic amino acid sequence near carboxy terminal generally extended by virus's lipid envelope, it is suitable to be used as helper peptide which can effectively deliver protein into a cell by promoting combination with lipid bilayer of liposome. Also, it was reported that if HA2 and PTD were used simultaneously, the function of secreting heterologous molecules, such as polypeptide, protein, etc. from endosome into cytoplasm, nucleus or other cellular organelles was enhanced, so that it results in promoting transmission into cells (US 2006/0222657A1).

diINF-7 domain derived from influenza virus, TLM (translocation motif) derived from Hepatitis B virus (HBV), L2 domain derived from Human Papilloma Virus (HPV) Histatin 5 domain developed as antibiotics, dhvar4 domain and dhvar5 domain which is synthetic peptides are known as peptides having similar function to HA2 (Stoeckl, L. et al., Proc. Natl. Acad. Sci., 103:6730, 2006; Kamper, N., J. Virol., 80:759, 2006; Mastrobattista, E. et al., J. Biol. Chem., 277:27135, 2002; den S Hertog, A. L. et al., Biochem. J., 379:665, 2004). They have the function of endosomal escape (endosomal rescue) that viruses or peptides introduced into cell perforate through lipid layer to be isolated into cytoplasm.

In the case of using PTD, it is known that comparatively large protein can be delivered into cells without a receptor, and it is expected to be refolded into protein having biological activity by HSPs (heat shock proteins), and the like (Nature Med., 4:1449, 1998; Science, 285:1549, 1999). However, methods of effectively delivering proteins to cells and tissues encounter various technical problems. Recently, a method for effectively delivering proteins into cells was suggested, which uses a protein transduction domain (PTD) present in HIV (Nature Med., 4:1449, 1998; Science, 285:1569, 1999). However, in the case of secreted proteins such as sFRP or DKK group, even if the proteins are effectively delivered into cells, whether activated antagonists are secreted and also can function to inhibit Wnt signaling is not completely known, and for this, experimental demonstration for the following procedures is required: (1) the intracellular introduction of a PTD fusion protein and the resulting activated protein secretion; (2) an appropriate intracellular activation process of the introduced protein using PTD, because proteins secreted in vivo undergo cleavage by proprotein convertase, such as furin in cells, and activation processes, such as glycosylation; and (3) verification on whether the secreted protein shows an appropriate biological function.

Many studies on clinical therapeutic effects resulting from the use of Wnt antagonists are not yet conducted. Currently commercially available Wnt antagonists are used for research purpose only, and the antagonists include DKK-1 derived from insect cells (sf21), DKK-4 and sFRP-4, derived from animal cells, due to the problem of post-translational modification as described above. However, these antagonists have very high production cost, because they are produced by isolating and purifying active proteins secreted into media containing transformed cells, and these antagonists are difficult to produce in large quantities, because the loss of their activity during an extraction process is inevitable.

Thus, in the art to which the present invention pertains, there is an urgent need to develop a novel biochemical substance which has at least the same biomedical utility as that of said sFRPs or DKKs and, at the same time, can be prepared at a cost much lower than that of previously known proteins and can fundamentally solve various problems, including inefficient inconveniency of isolation and purification steps, inconvenience in storage, handling and administration steps, and a reduction in activity.

Accordingly, the present inventors have made extensive efforts to develop a novel formulation capable of effectively treating Wnt signaling-mediated diseases such as cancer metastasis or rheumatoid arthritis. As a result, the present inventors have paid attention to non-activated Wnt inhibition polypeptides (WIPs) containing a protein transduction domain (PTD) which enables WIPs to permeate a cell membrane without cell-membrane receptor, and a Wnt antagonist domain (WAD) which is inactive by itself, but is activated in mammalian cells and then secreted out of the cells to function to inhibit Wnt signaling. The present inventors also found that said non-activated WIPs are prepared at costs significantly lower than those of prior active proteins, are easy to store and handle, and are very simple to administer. The inventors also found that said non-activated WIPs have a new pharmacological mechanism in which they permeate into cells without a receptor, are activated by HSP (heat shock protein), and the like in the cells, and inhibit Wnt signaling through the secretion of a large amount of the activated proteins. Thereby the present inventors completed the present invention.

### SUMMARY OF INVENTION

It is a main object of the present invention to provide non-activated Wnt inhibition polypeptides (WIPs), which have the structures, characteristics and pharmacological mechanisms completely different from those of previously known active proteins and are designed such that they can be administered directly to the human body to inhibit a Wnt signaling system so as to either inhibit the growth and metastasis of epithelium-derived cancer cells or treat Wnt signaling-mediated diseases such as rheumatoid arthritis, as well as a preparation method thereof.

Another object of the present invention is to provide a pharmaceutical composition containing said non-activated WIPs as an active ingredient.

To achieve the above objects, in one aspect, the present invention provides a non-activated Wnt inhibition polypeptide (WIP) containing: (a) a protein transduction domain (PTD) which enables said WIP to permeate a cell membrane without cell-membrane receptor; and (b) a Wnt antagonist domain (WAD) which is inactive by itself, but is activated in mammalian cells and then secreted out of the cells to function to inhibit Wnt signaling.

The non-activated Wnt inhibition polypeptide (WIP) according to the present invention preferably additionally comprises (c) EED (Endosomal escape domain) to avoid a decrease in biological activity by macropinosome or endosome and increase cell transduction rate.

In another aspect, the present invention provides a recombinant vector, wherein a base sequence of PTD, a base sequence for tagging, and a base sequence encoding at least four histidines for isolation and purification, are inserted upstream of the 5′-region of DNA encoding a Wnt antagonist domain (WAD) selected from the group consisting of sFRP-1 (FRP, SARP2, FrzA), sFRP-2 (SARP1), sFRP-3 (FzB, Fritz), sFRP-4 (FrzB-2), sFRP-5 (SARP3), Sizzled, Sizzled2, Crescent, WIF-1, Cerberus, Coco, DKK-1, DKK-2, DKK-3 (REIC), DKK-4, and Soggy (DKKL2), as well as bacteria transformed with said recombinant vector.

The recombinant vector according to the present invention additionally comprises a base sequence encoding EED (Endosomal escape domain) capable of avoiding a decrease in biological activity by macropinosome or endosome, and capable of increasing cell permeability.

In still another aspect, the present invention provides a method for preparing a non-activated Wnt inhibition polypeptide (WIP), the method comprising the steps of: (a) expressing PTD-WAD fusion polypeptide or PTD-EED-WAD fusion polypeptide by culturing said bacteria transformed with the recombinant vector; (b) denaturing two-dimensional and three-dimensional structures of the polypeptide or changing the two-dimensional and three-dimensional structures of polypeptide into a one-dimensional linear structure by recovering the cell pellets from the culture broth to add urea solution to the resulting cell pellets; and (c) purifying the denatured PTD-WAD fusion polypeptide or the PTD-EED-WAD fusion polypeptide (non-activated WIP or denatured WIP).

In yet another aspect, the present invention provides a pharmaceutical composition for inhibiting the growth and metastasis of cancer cells, which contains said non-activated WIP as an active ingredient. Also, the present invention provides a pharmaceutical composition for the treatment of immune diseases and inflammation, which contains said non-activated WIP as an active ingredient. Moreover, the present invention provides a pharmaceutical composition for the inhibition of pulmonary fibrosis, which contains said non-activated WIP as an active ingredient.

Other features and embodiments of the present invention will be more fully understood from the following detailed description and the appended claims.

### BRIEF DESCRIPTION OF DRAWINGS

FIG. 1 shows that the expression of Snail is inhibited due to the expression of human DKK-1 (hDKK-1).

FIG. 2 is an electrophoresis photograph of a total protein, before and after protein expression is induced with IPTG during the culture of transformed E. coli BL21.

FIG. 3 shows the result of Western blot of TAT-DKK-1, conducted using an anti-X-press antibody.

FIG. 4 shows the result of a test conducted to examine whether TAT-DKK-1 according to the present invention increases E-cadherin promoter activity in cells.

FIG. 5 shows the effect of the inventive TAT-DKK-1 on the inhibition of cancer cell invasion.

FIG. 6 shows the result of immunoprecipitation conducted on active DKK-1 secreted into a cell culture medium after TAT-DKK-1 and TAT-HA2-DKK-1 according to the present invention were administered.

### DETAILED DESCRIPTION OF THE INVENTION, AND PREFERRED EMBODIMENTS

In one aspect, the present invention relates to a non-activated Wnt inhibition polypeptide containing: (a) a protein transduction domain (PTD) which enables the polypeptide to permeate a cell membrane without cell-membrane receptor; and (b) a Wnt antagonist domain (WAD) which is inactive by itself, but is activated in mammalian cells and then secreted out of the cells to function to inhibit Wnt signaling.

The prior preparation of active DKKs or sFRPs has a problem in that it must depend on the culture of recombinant animal cells, such as sf21 or NSO having remarkably low productivity. Recombinant proteins, such as DKK-1, DKK-4 and sFRP-4, which are commercially available from R&D Systems Co., have problems in that they are isolated and purified through a complicated process at high cost. Also, some recombinant proteins are prepared through the culture of inexpensive recombinant E. coli strains which are generally used, but this preparation imposes many limitations on the biochemical structure and activity of secreted proteins to be prepared.

The present inventors have paid attention to a non-activated polypeptide which has no biochemical activity, i.e. a three-dimensional steric structure, primarily giving inefficiency in administering Wnt antagonists to the human body or mammal body. The present inventors have tried to make the non-activated polypeptide to be able to permeate into the cell membrane without receptor. As a result, the fusion polypeptide of TAT-DKK-1 according to the present invention was introduced into cells through the cell membrane. The non-activated TAT-DKK-1 inhibited Wnt signaling to reduce the expression of the Snail gene, thus it resulting in inhibiting invasion of cancer cells.

In the present invention, said WAD is not specifically limited, as long as said WAD, such as sFRP-1 (FRP, SARP2, FrzA), sFRP-2 (SARP1), sFRP-3 (FzB, Fritz), sFRP-4 (FrzB-2), sFRP-5 (SARP3), Sizzled, Sizzled2, Crescent, WIF-1, Cerberus, Coco, DKK-1, DKK-2, DKK-3 (REIC), DKK-4, Soggy (DKKL2) permeates into cells to be activated by HSP, and the like in cells, followed by being secreted out of the cells to inhibit Wnt signaling. Particularly, cystein-rich domain recombinant existing in most of these genes may be effective to inhibit specific signal delivery (Brott, B. K. & Sokol, S. Y., Mol. Cell Biol., 22:6100, 2002).

Although non-activated Wnt inhibition polypeptide (WIP) according to the present invention preferably contain PTD and WAD, it is more preferable to contain EED (Endosomal Escape domain), such as HA2 domain capable of avoiding a decrease in the biological activity by macropinosome or endosome and capable of increasing cell transduction rate. In other words, if WIP contains EED, such as HA2 domain, the non-activated Wnt inhibition polypeptide can overcome the problem where it is limited within endosome or macropinosome after it permeates a cell membrane (Jehangir, S. W. et al., Nature Med., 10:310, 2004).

In the present invention, HA2 peptide derived from influenza virus was used as EED, but it is not limited thereto. For example, reagents, such as chloroquine or sucrose enabling cell permeable peptides to avoid to be embedded into the lipid bilayer, diINF-7 domain derived from influenza virus, TLM (translocation motif) derived from Hepatitis B virus (HBV), L2 domain derived from human papilloma virus (HPV), Histatin 5 domain developed as antibiotics, dhvar4 domain and dhvar5 domain which are synthetic peptides may be used as EED (Stoeckl, L. et al., Proc. Natl. Acad. Sci., 103:6730, 2006; Kamper, N., J. Virol., 80:759, 2006; Mastrobattista, E. et al., J. Biol. Chem., 277:27135, 2002; den Hertog, A. L. et al., Biochem. J., 379:665, 2004).

More specifically, HA2 domain (GLFGAIAGFIENGWEGMIDG: SEQ ID NO: 4), HA2 domain analogue (GLFEAIAEFIEGGWEGLIEG: SEQ ID NO: 5), diINF-7 domain (GLFEAIEGFIENGWEGMIDG: SEQ ID NO: 6), TLM1 (LLNQLAGRMIPK: SEQ ID NO: 7), TLM2 (TLDHVLDHVQTM: SEQ ID NO: 8), HPV3 3L2 (SYFILRRRRKRFPY: SEQ ID NO: 9), HPV1 6L2 (SYYMLRKRRRKRLPY: SEQ ID NO: 10), HPV1 8L2 (LYYFIRKKRKRVPY: SEQ ID NO: 11), Histatin 5 (DSHAKRHHGYKRKFHEKHHSHRGY: SEQ ID NO: 12), dhvar4 (KRLFKKLLFSLRKY: SEQ ID NO: 13), or dhvar5 (LLLFLLKKRKKRKY: SEQ ID NO: 14) and a complex with a combination of at least two thereof, may be used.

The non-activated WIPs according to the present invention are polypeptides containing: PTD which enables said WIPs to permeate a cell membrane without cell membrane receptor; WAD which can be activated by HSP, and the like in cells and secreted out of the cells to inhibit Wnt signaling and selectively, EED capable of avoiding a decrease in the biological activity by macropinosome or endosome and increasing cell transduction rate. Specifically, the non-activated WIPs according to the present invention are inactive by themselves, but they are activated after they permeate organisms or cells, and the activated proteins are secreted out of the cells to show potency. Such non-activated WIPs according to the present invention can solve all the disadvantages of the prior activated proteins. The disadvantages of the prior activated proteins are that they are prepared at high cost, are not easy to be stored and handled, and that they necessarily need a receptor.

In another aspect, the present invention relates to a recombinant vector, wherein a base sequence of PTD, a base sequence for tagging, and a base sequence encoding at least 4 histidines for isolation and purification, are inserted upstream of the 5′-region of DNA encoding Wnt antagonist domain (WAD). The WAD according to the present invention is any one or combination of more than 2 selected from the group consisting of sFRP-1 (FRP, SARP2, FrzA), sFRP-2 (SARP1), sFRP-3 (FzB, Fritz), sFRP-4 (FrzB-2), sFRP-5 (SARP3), Sizzled, Sizzled2, Crescent, WIF-1, Cerberus, Coco, DKK-1, DKK-2, DKK-3 (REIC), DKK-4, and Soggy (DKKL2), as well as bacteria transformed with said recombinant vector.

In the present invention, a base sequence for tagging, a base sequence encoding at least four histidines for isolation and purification, and ATG as a start base sequence, were inserted upstream of the 5′-region of DKK-1 gene, thus constructing a recombinant vector. The recombinant vector according to the present invention preferably additionally comprises a base sequence encoding EED described above.

In the present invention, a gene encoding DKK-1 was used as the Wnt signaling-inhibiting gene, but it is not limited thereto. Also, TAT (YGRKKRRQRRR) was used as said PTD, but the present invention is not limited thereto.

For example, PTD, such as AntHD (Drosophila homeoprotein atennapedia transcription protein; RQIKIWFQNRRMKWKK: SEQ ID NO: 15), VP22 (Gene Therapy, 8:1, Blackbirch Press, 2001) peptide, mph-1-btm (US patent 2005/0147971), poly-Arg (RRRRRRR: SEQ ID NO: 16), PTD-5 (RRQRRTSKLMKR: SEQ ID NO: 17), transportan (PQIKIWFQNRRMKWKK: SEQ ID NO: 19), Buforin II (TRSSRAGLQFPVGRVHRLLRK: SEQ ID NO: 20), Ku 70 (VPMLK-PMLKE: SEQ ID NO: 21), SynB1 (RGGRLSYSTTTFSTSTGR: SEQ ID NO: 22), Pep-1 (KETWWETWWTEWSQPKKKRKV: SEQ ID NO: 23), Pep-7 (SDLWEMMMVSLACQY: SEQ ID NO: 24), HN-1 (TSPLNIHNGQKL: SEQ ID NO: 25) may be used in the present invention (Joliot, A. & Prochiantz, Nature Cell Biol., 6:198, 2004; Kabouridis, P. S., Trends Biotechnol., 21:498, 2003). Moreover, an X-press tag was used as said tagging base sequence, but Flag, Myc, Ha, GST, or the like may also be used in the present invention

In the present invention, commercially available ampicillin-resistant pRSET was used to construct said recombinant vector, but the present invention is not limited thereto. For example, a bacterial vector having kanamycin as a selective marker, mammalian cell expression vector such as pcDNA, or virus vector such as pPGS or pBabe may also be used.

In still another aspect, the present invention relates to a method for preparing a non-activated Wnt inhibition polypeptide (WIP), the method comprising the steps of: (a) expressing PTD-WAD fusion polypeptide by culturing bacteria transformed with a recombinant vector containing PTD base sequence and DNA encoding WAD; (b) denaturing two-dimensional and three-dimensional structures of the PTD-WAD fusion polypeptide or changing the two-dimensional and three-dimensional structures of PTD-WAD fusion polypeptide into a one-dimensional linear structure by recovering the cell pellets from the culture broth to add urea solution to the resulting cell pellets; and (c) purifying the denatured PTD-WAD fusion polypeptide (non-activated WIP).

In still another aspect, the present invention relates to a method for preparing a non-activated Wnt inhibition polypeptide (WIP), the method comprising the steps of: (a) expressing PTD-EED-WAD fusion polypeptide by culturing bacteria transformed with a recombinant vector containing PTD base sequence, a base sequence encoding EED and DNA encoding WAD; (b) denaturing two-dimensional and three-dimensional structures of the PTD-EED-WAD fusion polypeptide or changing the two-dimensional and three-dimensional structures of PTD-EED-WAD fusion polypeptide into a one-dimensional linear structure by recovering the cell pellets from the culture broth to add urea solution to the resulting cell pellets; and (c) purifying the denatured PTD-EED-WAD fusion polypeptide (WIP).

In the inventive method for preparing the non-activated WIP, the purification step may preferably comprise: (a) binding the polypeptide to nickel-titanium beads; (b) washing the polypeptide-bound beads with urea solution; and then (c) eluting the polypeptide using imidazole and buffer containing salt, but it is not limited thereto.

The microorganism transformed with said recombinant vector was cultured to express the non-activated WIPs according to the present invention, and then the expressed proteins were isolated and purified. In the present invention, the transformed E. coli can be cultured in a conventionally used medium, and IPTG is preferably added thereto in order to induce the over-expression of the fusion polypeptide.

Although E. coli was primarily used as the transformed microorganisms in the present invention, it is also possible to use other kinds of bacteria, yeasts, or fungi. Furthermore, it is also possible to use only active sites synthesized through a chemical method, without using microorganisms.

The fusion polypeptide expressed by the culture of the transformed microorganism can be isolated using a method for the isolation and purification of GST-fusion proteins or other conventional proteins. For example, the inventive non-activated WIPs can be purified by inducing the precipitation of protein using a concentration gradient of urea or ammonium sulfate and dialyzing the precipitated substance to remove salts. In the present invention, the over-expressed polypeptide is preferably used after denaturing the two-dimensional and three-dimensional structures thereof into an inactive, one-dimensional linear structure using urea, because the two-dimensional and three-dimensional structures thereof are unnecessary. However, if the polypeptide contains functional domain described in the present invention, the polypeptide with two-dimensional and three-dimensional structures can also penetrate into cells and thus show similar effect.

Although it is composed in the order of Histidine tag for isolating and purifying, X-press tag for examination of expression, PTD for penetrating cell membrane, EED (Endosomal escape domain) to avoid a decrease in the biological activity by endosome, and WAD for inhibiting Wnt signal in the present invention,, another type of recombination in another order thereof may also show similar effect if each functional domain is fully included. Also, as described in WO 2006/104306A1, since cleavage by proprotein convertase and protein modification result in the activity of a secretory protein, an increase in the physiological activity can be expected if furin cleavage site occurs naturally or is artificially added for an increase in the biological activity. Especially, it is known that, in the case of sFRPs and DKKs, putative furin cleavage sites exist, but activation process by furin is seldom known (Kawano, Y. & Kypta, R., J. Cell Sci., 116:2627, 2003).

The non-activated WIPs according to the present invention can be produced in large quantities through the culture of bacteria, such as recombinant E. coli, and they are biochemically inactive before being administered into the human body. Therefore the production cost thereof is only one several tenths to one several hundredth of that of the previously known active proteins having uses similar thereto, and the isolation/purification and handling/administration processes thereof are significantly simple and convenient.

In yet another aspect, the present invention relates to a pharmaceutical composition for inhibiting the growth and metastasis of cancer cells, which contains said non-activated WIP as an active ingredient. Also, the present invention relates to a pharmaceutical composition for the treatment of immune disease, which contains said non-activated WIP as an active ingredient. In the present invention, said immune disease is preferably arthritis. Moreover, the present invention relates to a pharmaceutical composition for the inhibition of pulmonary fibrosis, which contains said non-activated WIP as an active ingredient.

The non-activated WIPs according to the present invention have no three-dimensional stereo-regularity and are inactive by themselves, but are activated by HSP and the like after they permeate into cells, and the activated proteins are secreted out of the cells to show potency. The non-activated WIPs according to the present invention inhibit the growth and metastasis of epithelium-derived cancer cells, and inhibit the progression of immune diseases such as rheumatoid arthritis, and furthermore, they have therapeutic effects.

The present invention fundamentally solves the problems of high cost and the inefficiency of isolation, purification, storage and administration of the active products, which has often arisen in the previously known active recombinant proteins. In addition, it was found that the non-activated polypeptides can be administered directly into the human body or mammalian body and, at the same time, they show the effect of inhibiting Wnt signaling.

If the inventive non-activated WIPs are administered to cancer cells, progenitor cells, activated FLS (fibroblast-like synoviocytes), stem cells, and the like, the non-activated WIPs will be reconstructed by HSP, and the like and they will be secreted through an activation process such as N-glycosylation. Herein, the non-activated WIPs, which permeated into the cells, have a half-life of 3-24 hours depending on the kind and activity of the cells, so that it suggests that the permeated proteins have an activation time which varies depending on the kind of cells.

If the non-activated WIPs purified in the present invention is administered to cells at a concentration higher than 0.1 nM, the non-activated WIPs will be transported into the cells in a concentration-dependent manner, and they will be converted into activated DKK, and the like in the cells, and then they will be secreted out of the cells in the form of biologically active DKK. Most of the inventive non-activated WIPs directly permeated the cell membrane of cells within 1 hour without regard to whether the Fz receptor or the LRP5/6 co-receptor was present in the cell membrane, and the membrane permeation process was temperature-independent without the receptor.

Accordingly, the non-activated WIPs according to the present invention do not need to maintain a three-dimensional structure, and they show a mechanism in which they permeate into cells in the state of a one-dimensional linear structure, are converted into active Wnt antagonists, and the like in the cells, and then secreted out of the cells. In other words, the prior DKK and sFRP directly show potency, whereas the non-activated WIPs according to the present invention indirectly show potency. Thus, the inventive non-activated WIPs do not require an additional system or cost for maintaining the three-dimensional structure thereof, are very easy to isolate and purify, are produced through a simple process, and have low production cost and increased medical efficiency, so that it suggests that the inventive non-activated WIPs can solve all the problems that the prior recombinant proteins have. The characteristics of the non-activated WIPs according to the present invention are shown in Table 1 below in comparison with those of prior recombinant Wnt antagonist proteins. Table 1 shows the comparison between the inventive non-active WIPs and the previously known DKKs and sFRPs with respect to processes for the isolation, purification, storage and administration thereof, and the characteristics thereof.

TABLE 1
Comparison of characteristics of Prior recombinant
Wnt antagonist protein and non-activated WIPs

| | Prior Wnt antagonists | |
|---|---|---|
| Items | (DKK-1, DKK-4, SFRP-4) | Inventive non-activated WIPs |
| Production method | Culture of transformed sf21, NSO | Culture of transformed |
| | cells | E. coli |
| | Produce while maintaining the | Maintain the transformed |
| | transformed cells | E. coli in a simple manner |
| | Separate from large cell culture | Separate directly from |
| | and concentrate | E. coli, and dilute |
| Production cost | Very high | Significantly lower than |
| | | those of the prior art |
| Production equipment and | Require large-scale equipment | Very simple |
| facilities | and facilities | |
| Products | Activated DKK or sFRP | Fusion polypeptides of |
| | | PTD-DKK or PTD-sFRP |
| Protein three-dimensional | Active three-dimensional structure | Non-activated or denatured |
| structure | | peptides |
| | Naturally occurring structure | Naturally non-occurring |
| | | random structure |
| Solubility in cleaning | Soluble; | Insoluble; |
| material and physiological | Loss of three-dimensional structure | No effect on structure and |
| saline | and activity | activity |
| Need of carrier for | Diffuse rapidly due to water | Are insoluble and |
| administration in vivo | solubility. | transducible rapidly into |
| | Need suitable carrier for local | adjuacent cells. |
| | administration | No need of carrier for local |
| | | administration |
| Stability of storage | Loss of activity upon breakdown of | Independent on structure; |
| | three-dimensional structure; | Storable above 37□; |
| | Not storable above 37□; | Good stability of storage |
| | Low stability of storage | |
| Biological mechanism | Directly bind to cell membrane | Activated proteins are |
| | receptors (direct action) | secreted out of cells |
| | | (indirect action) |
| Temperature dependency | Needs living body temperature to be | Permeate all living cells |
| in administration | bound to receptors | independently of temperature |
| Half-life for activation | No relevant data | 3-12 hours |
| Function of signal peptide | Necessary | Not necessary |
| in process for protein | | |
| activation | | |
| Cell selectivity | Signal only through Fz receptors | Permeable all kinds of cells |
| | and LRP5/6 aid-receptors | without help from receptors |

| | |
|---|---|
| Medical potency | Similar |
| Administration mode | Same |

The non-activated WIPs according to the present invention can be used to treat Wnt signaling-mediated diseases or inhibit the progression thereof, for example, to inhibit the proliferation and metastasis of epithelium-derived cancer cells, or inhibit the progression of rheumatoid arthritis and treat it. Also, they can be used to regulate the differentiation of stem cells and apply these cells to treat diseases.

The non-activated WIPs according to the present invention are used alone or used in the form of pharmaceutically acceptable acid addition salts or metal complexes thereof, for example, zinc or iron salts. Specifically, as the acid addition salts, it is preferable to use hydrogen chloride, hydrogen bromide, sulfates, phosphates, maleates, acetates, citrates, benzoates, succinates, malates, ascorbates or tartrates.

A composition containing the inventive non-activated WIPs as an active ingredient is preferably prepared by diluting the active ingredient with a pharmaceutically acceptable excipient or matrix carrier, or encapsulating the active ingredient into a container-shaped carrier, according to the administration method, the mode of administration and intended therapeutic use thereof.

The preparation of the physiologically acceptable protein composition, having due regard to pH, isotonicity, stability, and the like, may be the method within the skill of the art to which the present invention pertains. In the present invention, the choice of said matrix is based on biocompatibility, biodegradability, mechanical properties, cosmetic appearance and interface properties, and potential matrices for the composition may be biodegradable and chemically defined substances, such as calcium sulfate, tricalcium phosphate, hydroxyapatite, polylactic acid and polyanhydrides, biodegradable and biologically defined substances, such as dermal collagen, pure proteins or extracellular matrix components, non-biodegradable and chemically defined substances, such as sintered hydroxyapatite, bioglass, aluminates, or other ceramics, and combinations of any of the above mentioned types of material, such as polylactic acid and hydroxyapatite or collagen and tricalcium phosphate. However, it is not limited to the above-described carriers.

The excipient, which can be used in the present invention, may be lactose, dextrose, sucrose, sorbitol, mannitol, calcium silicate, cellulose, methyl cellulose, amorphous cellulose, polyvinylpyrrolidone, magnesium stearate, water, methylhydroxybenzoate, propylhydroxybenzoate, talc, mineral oil or the like.

Meanwhile, the composition containing the non-activated WIPs according to the present invention can be preferably used by encapsulating or injecting in viscous liquid form for delivery to the site in need of therapeutic effects. The dosage of the inventive composition can be determined considering the kind of carrier (e.g., excipient or matrix) used in the composition, the patient's conditions, the patient's age, sex, and diet, the severity of any infection, time of administration and other clinical factors.

### Examples

Hereinafter, the present invention will be described in detail with reference to specific examples. However, those skilled in the art will appreciate that these examples are not intended to limit the scope of the present invention, and various changes and modifications are possible within the sprit and scope of the present invention.

Particularly, in the following examples, only DKK-1 was illustrated as a gene of WAD constituting non-activated WIPs, but those skilled in the art will appreciate that it is possible to use a protein gene which is secreted out of cells to inhibit Wnt signaling, such as any one of genes or a combination of at least two genes selected from the group consisting of sFRP-1, sFRP-2, sFRP-3, sFRP-4, sFRP-5, Sizzled, Sizzled-2, Crescent, WIF-1, Cerberus, Coco, DKK-1, DKK-3, DKK-4, Soggy, or the like. In addition, those skilled in the art will also understand that it is possible to use not only human Wnt antagonists, but also Wnt antagonists derived from mammals, such as rats, cattle and pigs, and other higher animals.

In the following examples, only TAT was illustrated as PTD, but those skilled in the art will appreciate that it is possible to use a gene selected from the group consisting of AntHD (Drosophila homeoprotein atennapedia transcription protein),

VP22 peptide, mph-1-btm, Penetratin, Buforin II, Transportan, Ku70, Prion, Pvec, and Pep-1. Also, in the following examples, only HA2 was illustrated as EED, but those skilled in the art will appreciate that it is possible to use any one of genes or a combination of at least two genes selected from the group consisting of diINF-7 domain, TLM1 domain, TLM2 domain, HPV3 3L2 domain, HPV1 6L2 domain, HPV1 8L2 domain, Histatin 5 domain, dhvar4 domain and dhvar5 domain.

### Example 1

### Inhibition of Snail Expression By Use of DKK-1 Expression Vector

Wnt signaling induces the activation of Snail through a mechanism of inhibiting GSK3-β. Meanwhile, Snail is known to inhibit the transcription of E-cadherin and regulate the migration/metastasis of epithelium-derived cells (Nature Cell Biol., 2:84, 2000; Nature Cell Biol., 2:76, 2000; Nature Rev. Mol. Cell Biol., 3:155, 2002). Thus, Wnt signaling inhibits the expression of E-cadherin via Snail and also induces the invasive growth and metastasis of cancer cells. Therefore, if the expression of Snail, which is regulated by Wnt, can be inhibited, the invasive growth and metastasis of cancer cells can be inhibited.

In this Example, a gene encoding hDKK-1 was amplified by RT-PCR using 293 cell (American Type Culture Collection, ATCC CRL-1573) mRNA as a template with primers of SEQ ID NOs: 1 and 2.

| | |
|---|---|
| 5′-gct tgc aaa gtg acg gtc att-3′ | (SEQ ID NO: 1) |
| 5′-cta tcc aaa tgc agt gaa ctc-3′ | (SEQ ID NO: 2) |

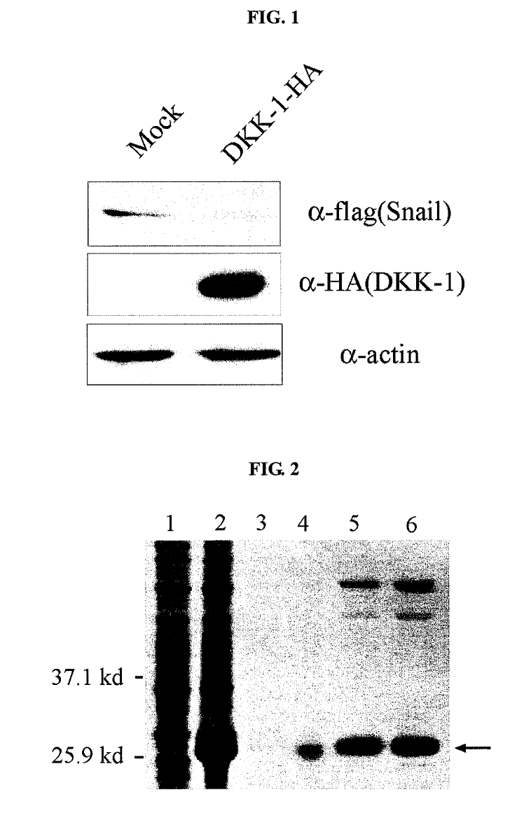

The amplified gene was inserted into a TA cloning vector (Invitrogen, Inc) and compared with the base sequence (GenBank, NM_{—}012242) of GenBank and, as a result, found to be identical to the hDKK-1 gene. The cloned hDKK-1 gene was further cloned into the BamHI and EcoRI sites of a pCR3.1 expression vector (mammalian expression vector; Invitrogen, Inc.), and an HA tag for examination of expression was inserted in front of the stop codon of the hDKK-1 gene. The expression of the completed expression vector together with the Snail gene was induced in the 293 cells using lipofectamine (Invitrogen). The Snail gene was tagged with a flag, the expression of Snail and the expression of DKK were verified by Western blot using an anti-flag antibody (Sigma) and an anti-HA antibody (Roche), respectively (FIG. 1).

As a result, as shown in FIG. 1, it could be seen that inhibition of the Wnt signaling system through the expression of hDKK-1 reduces the half-life of Snail to reduce the expression of the Snail gene, and this result was consistent with the mechanism of the prior literature (J. Biol. Chem., 280:11740, 2005).

### Example 2

### Inhibition of Wnt Signaling and Cancer Cell Invasion By TAT-DKK-1

The hDKK-1 gene cloned in Example 1 was further cloned into bacterial expression vector pRSET (Invtrogen). TAT (YGRKKRRQRRR: SEQ ID NO: 3) was inserted in front of the 5′-region of the hDKK-1 gene, and then X-press (Invitrogen, Inc.) tag, a base sequence encoding 6 histidines for isolation and purification, and start base sequence ATG, were inserted in front thereof, thus constructing a recombinant expression vector for the expression of hDKK-1.

The constructed recombinant vector was introduced into E. coli BL21 (Invitrogen Inc.) using a heat shock method, and the bacterial cells were cultured at 37□ for 2-3 hours. Then, IPTG (isopropylthio-galactoside) was added thereto to a concentration of 1 mM, and the bacterial cells were additionally cultured for 2-18 hours to induce the expression of TAT-DKK-1.

Said E. coli were centrifuged from the culture broth, and cell pellets were collected from the culture broth. An 8M urea solution was added to the cell pellets to remove two-dimensional and three-dimensional structures from the DKK-1 gene, and then Ni—Ti beads (Qiagen) were added to the cell pellets so as to link TAT-DKK-1 to the beads. Then, the beads were washed three times with the urea solution to elute with imidazole and buffer containing salt, thus obtaining purified TAT-DKK-1 fusion polypeptide (FIG. 2).

FIG. 2 shows the result of electrophoresis conducted to confirm the purification process of the protein after inducing protein expression by IPTG during the culture of transformed E. coli BL21. In FIG. 2, the arrow indicates the TAT-DKK-1 fusion protein induced by IPTG, lane 1 indicates uninduced TAT-DKK-1, lane 2 indicates TAT-DKK-1 induced by IPTG, and lanes 3-6 indicate the results of elution conducted by gradually increasing concentrations of imidazole during the separation of TAT-DKK-1 from the Ni—Ti beads. As shown in FIG. 2, it can be observed that, in the case where the production of the fusion protein was induced by the addition of IPTG, the TAT-DKK-1 fusion protein was produced in an amount larger than that in a control group, and when the concentration of imidazole was increased during the separation of the fusion protein, the TAT-DKK-1 fusion protein was eluted in an increased amount.

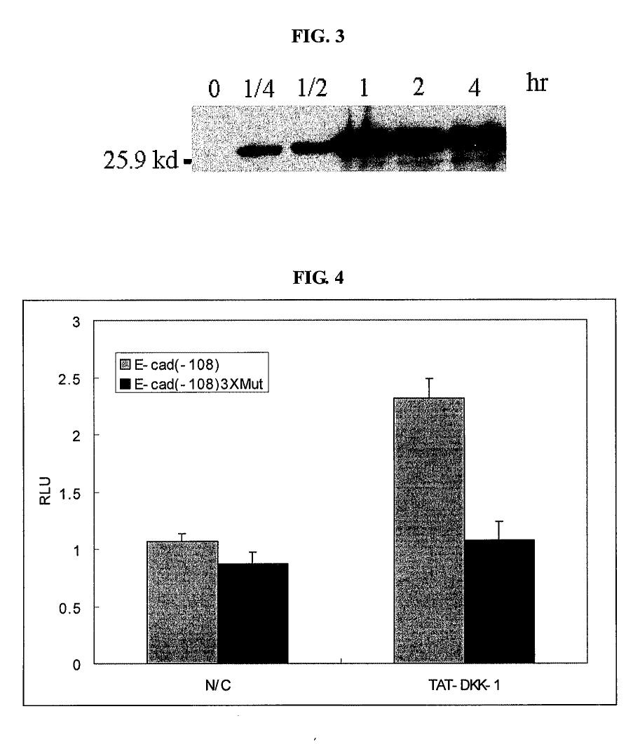

Meanwhile, in order to examine whether the above-prepared TAT-DKK-1 permeates into cells, the MCF-7 breast cancer cell line (ATCC, HTB-22) was treated with 4 nM TAT-DKK-L for 2 hours, and then the cells were collected and subjected to Western blot using an anti-X-press primary antibody (Invitrogen, Inc) (FIG. 3).

As a result, as shown in FIG. 3, it could be seen that most of TAT-DKK-1 permeated into cells within 4 hours. In FIG. 3, lanes 1-6 indicate the results of Western blot conducted to examine the expression of TAT-DKK-1 in the cells using the anti-X-press antibody after 0 hr, 15 minutes, 30 minutes, 1 hour, 2 hours and 4 hours of treatment with 4 nM TAT-DKK-1, respectively.

In order to examine whether TAT-DKK-1 introduced into cells is converted into biologically active DKK-1, and the converted DKK-1 inhibits Wnt signaling to inhibit Snail expression, and ultimately to induce an increase in the expression of E-cadherin, a luciferase reporter gene assay with an E-cadherin proximal promoter was conducted (J. Biol. Chem., 280:11740, 2005). MCF-7 cells were transfected with said reporter gene using Fugene-6, and after 24 hours, administered with 2 nM TAT-DKK-1 for 24 hours, and the E-cadherin promoter activity was measured using luciferase activity. E-cad(-108)3XMut having a mutation in the Snail-binding site of the E-cadherin promoter was used as a negative control group (FIG. 4).

As a result, as shown in FIG. 4, it could be observed that TAT-DKK-1 according to the present invention inhibited the expression of Snail as described in Example 1 above, and activated the E-cadherin promoter, thus effectively inhibiting Wnt signaling.

### Example 3

### Inhibition of Wnt Signaling and Cancer Cell Invasion by TAT-DKK-1

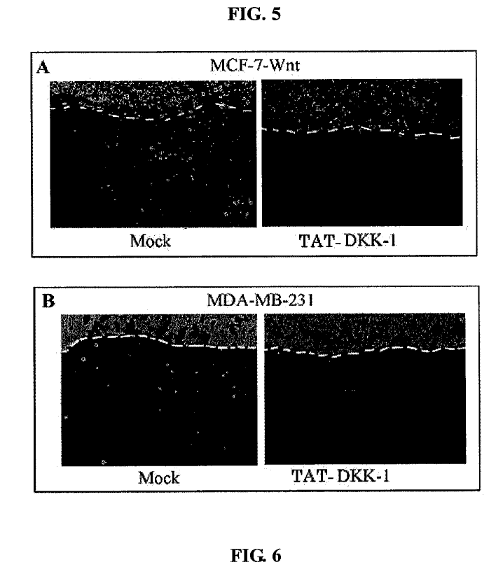

In order to examine whether the non-activated TAT-DKK-1 according to the present invention inhibits the invasive growth of cancer cells and, furthermore, can be used as a metastasis inhibitor, the expression of Wnt-1 in MCF-7 cells was induced (J. Biol. Chem., 280:11740, 2005). MCF-7 having no invasive ability shows invasive growth through Wnt expression, during which the invasion of cancer cells completely depends on Snail expression and E-cadherin promoter activity (J. Biol. Chem., 280:11740, 2005). Thus, if TAT-DKK-1 inhibits Wnt signaling, invasive growth induced by Wnt signaling can be inhibited. To demonstrate this, in this Example, the Wnt-expressing MCF-7 cell line was treated with 5 nM TAT-DKK-1 and cultured in the chorioalantoic membrane of chicken eggs for 48 hours, and the invasion of the cancer cells was observed with a fluorescent microscope (FIG. 5).

As a result, as shown in FIG. 5, TAT-DKK-1 completely blocked the invasive growth of the cancer cells, induced by Wnt signaling. Furthermore, the non-activated TAT-DKK-1 according to the present invention strongly inhibited invasive growth even in the MDA-MB-231 (ATCC HTB-26) cell line, which is known to have a very strong invasive ability due to the absence of E-cadherin expression. From these results, it could be found that the non-activated TAT-DKK-1 according to the present invention strongly inhibits the growth and metastasis of cancer cells.

### Example 4

### Preparation of HA2-TAT-DKK-1

After a sequence encoding HA2 domain (SEQ ID NO: 4) as typical EED was inserted at the end of TAT base sequence of the recombinant vector constructed in Example 2 to construct a recombinant vector, transformed E. coli was constructed, cultured and purified using the same method as in Example 2 to construct TAT-HA2-DKK-1 fusion polypeptide. The constructed TAT-HA2-DKK-1 fusion polypeptide and TAT-DKK-1 fusion polypeptide were treated with 293 cell line at the concentration of 10 nM for 2 hours and additionally cultured for 6 hours. Then, active DKK-1 secreted in a culture medium was examined using anti-HA immunoprecipitation. At this time, recombinant protein without TAT coding sequence was used as a negative control (N/C).

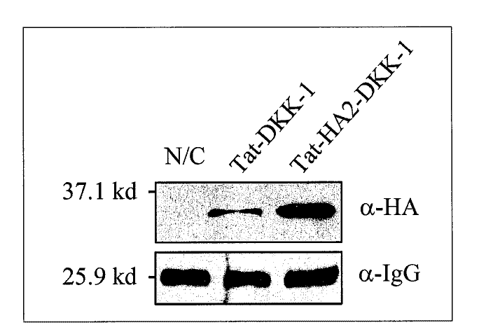

As a result, it was confirmed that, in the case where TAT-DKK-1 and TAT-HA2-DKK-1 were administered, active DKK-1 was expressed into the cell medium, specifically, in the case where TAT-HA2-DKK-1 contains EED, more active proteins were secreted. Light chain of immunoglobulin of antibody used in immunoprecipitation was used as loading control (FIG. 6).

### INDUSTRIAL APPLICABILITY

As described above in detail, the present invention provides the non-activated WIPs containing: PTD which enables said WIPs to permeate a cell membrane without the aid of a cell membrane receptor; and WAD which is inactive by itself, but is activated in mammalian cells and then secreted out of the cells to function to inhibit Wnt signaling. Also, the present invention provides the method for preparing said WIPs, and the composition for inhibiting the growth and metastasis of cancer cells and the composition for treating immune diseases, which contain said non-activated WIPs as active ingredients.

WIPs according to the present invention do not need to maintain a three-dimensional structure for binding to a cell membrane receptor, permeate into cells in the state of a one-dimensional linear structure, are activated by HSP, and the like in the cells and then secreted out of the cells to show potency. Thus, the inventive WIPs do not require an additional system or cost for maintaining the three-dimensional structure. Also, because the inventive WIPs contain the histidine sequence, the isolation and purification thereof are very easy, the production process thereof is simple, and the production cost thereof is low. In addition, they have increased medical efficiency, and thus are useful as novel therapeutic proteins.

Although a specific embodiment of the present invention has been described in detail, those skilled in the art will appreciate that this description is merely a preferred embodiment and is not construed to limit the scope of the present invention. Thus, the substantial scope of the present invention will be defined by the accompanying claims and equivalents thereof.

1. A non-activated Wnt inhibition polypeptide (WIP) comprising: (a) a protein transduction domain (PTD) wherein said PTD enables said WIP to permeate a cell membrane without cell-membrane receptor; and (b) a Wnt antagonist domain (WAD) wherein said WAD is inactive by itself, but is activated in mammalian cells and then secreted out of the cells to function to inhibit Wnt signaling.

2. The non-activated Wnt inhibition polypeptide (WIP) according to claim 1, wherein additionally comprises (c) EED (Endosomal escape domain) capable of avoiding a decrease in the biological activity by macropinosome or endosome and increasing cell transduction rate.

3. The non-activated Wnt inhibition polypeptide (WIP) according to claim 2, wherein the EED is any one of genes or a combination of at least two genes selected from the group consisting of HA2 domain (SEQ ID NO: 4), diINF-7 domain (SEQ ID NO: 6), TLM1 (translocation motif1) domain (SEQ ID NO: 7), TLM2 domain (SEQ ID NO: 8), HPV3 3L2 domain (SEQ ID NO: 9), HPV1 6L2 domain (SEQ ID NO: 10), HPV1 8L2 domain (SEQ ID NO: 11), Histatin 5 domain (SEQ ID NO: 12), dhvar4 domain (SEQ ID NO: 13) and dhvar5 domain (SEQ ID NO: 14).

4. The non-activated Wnt inhibition polypeptide (WIP) according to claim 3, wherein the HA2 domain has an amino acid sequence selected from the group consisting of SEQ ID NO: 4 and SEQ ID NO: 5, the diINF-7 domain has an amino acid sequence of SEQ ID NO: 6, the TLM1 domain has an amino acid sequence of SEQ ID NO: 7, the TLM2 domain has an amino acid sequence of SEQ ID NO: 8, the HPV3 3L2 domain has an amino acid sequence of SEQ ID NO: 9, the HPV1 6L2 domain has an amino acid sequence of SEQ ID NO: 10, the HPV1 8L2 domain has an amino acid sequence of SEQ ID NO: 11, the Histatin 5 domain has an amino acid sequence of SEQ ID NO: 12, the dhvar4 domain has an amino acid sequence of SEQ ID NO: 13 and the dhvar5 domain has an amino acid sequence of SEQ ID NO: 14.

5. The non-activated Wnt inhibition polypeptide (WIP) according to claim 1, wherein the WAD is any one of genes or a combination of at least two genes selected from the group consisting of: sFRP-1 (secreted Frizzled-related protein-1, FRP, SARP2, FrzA), sFRP-2 (SARP1), sFRP-3 (FzB, Fritz), sFRP-4 (FrzB-2), sFRP-5 (SARP3), Sizzled, Sizzled2, Crescent, WIF-1, Cerberus, Coco, DKK-1 (Dickkopf-1), DKK-2, DKK-3 (REIC), DKK-4, and Soggy (DKKL2).

6. The non-activated Wnt inhibition polypeptide (WIP) according to claim 1, wherein the PTD is selected from the group consisting of: TAT (transactivator of transcription), AntHD (Drosophila homeoprotein atennapedia transcription protein) peptide, VP22 (virus protein22) peptide, mph-1-btm (mouse transcription inhibitory factor-1-biomolecule transduction mortif), Penetratin, Buforin II, Transportan, Ku70, Prion, pVEC and Pep-1.

7. The non-activated Wnt inhibition polypeptide (WIP) according to claim 1, wherein PTD is fused with WAD.

8. A recombinant vector, wherein said vector comprises a base sequence of PTD, a base sequence for tagging, and a base sequence encoding at least four histidines for isolation and purification, are inserted upstream of the 5 ′-region of DNA encoding a Wnt antagonist domain (WAD) selected from the group consisting of sFRP-1 (FRP, SARP2, FrzA), sFRP-2 (SARP1), sFRP-3 (FzB, Fritz), sFRP-4 (FrzB-2), sFRP-5 (SARP3), Sizzled, Sizzled2, Crescent, WIF-1, Cerberus, Coco, DKK-1, DKK-2, DKK-3 (REIC), DKK-4, and Soggy (DKKL2).

9. The recombinant vector according to claim 8, further comprising a base sequence encoding EED (Endosomal escape domain) wherein said EED is capable of avoiding a decrease in the biological activity by macropinosome or endosome, and increasing cell transduction rate.

10. The recombinant vector according to claim 9, wherein the EED is any one of genes or a combination of at least two genes selected from the group consisting of HA2 domain (SEQ ID NO: 4), diINF-7 domain (SEQ ID NO: 6), TLM1 (translocation motif1) domain (SEQ ID NO: 7), TLM2 domain (SEQ ID NO: 8), HPV3 3L2 domain (SEQ ID NO: 9), HPV1 6L2 domain (SEQ ID NO: 10, HPV1 8L2 domain (SEQ ID NO: 11), Histatin 5 domain (SEQ ID NO: 12), dhvar4 domain (SEQ ID NO: 13) and dhvar5 domain (SEQ ID NO: 14).

11. The recombinant vector according to claim 10, wherein the HA2 domain has an amino acid sequence selected from the group consisting of SEQ ID NO: 4 and SEQ ID NO: 5, the diINF-7 domain has an amino acid sequence of SEQ ID NO: 6, the TLM1 domain has an amino acid sequence of SEQ ID NO: 7, the TLM2 domain has an amino acid sequence of SEQ ID NO: 8, the HPV3 3L2 domain has an amino acid sequence of SEQ ID NO: 9, the HPV1 6L2 domain has an amino acid sequence of SEQ ID NO: 10, the HPV1 8L2 domain has an amino acid sequence of SEQ ID NO: 11, the Histatin 5 domain has an amino acid sequence of SEQ ID NO: 12, the dhvar4 domain has an amino acid sequence of SEQ ID NO: 13 and the dhvar5 domain has an amino acid sequence of SEQ ID NO: 14.

12. A transformed bacteria, wherein said bacteria is transformed with the recombinant vector of claim 8.

13. A method for preparing a non-activated Wnt inhibition polypeptide (WIP), the method comprising the steps of:
(a) expressing PTD-WAD fusion polypeptide by culturing said bacteria transformed with the recombinant vector of claim 8;
(b) denaturing two-dimensional and three-dimensional structures of the polypeptide or changing the two-dimensional and three-dimensional structures of polypeptide into a one-dimensional linear structure by recovering the cell pellets from the culture broth to add urea solution to the resulting cell pellets; and
(c) purifying the denatured PTD-WAD fusion polypeptide (WIP).

14. The method for preparing a non-activated Wnt inhibition polypeptide (WIP) according to claim 13, wherein the PTD is selected from the group consisting of: TAT (transactivator of transcription), AntHD (Drosophila homeoprotein atennapedia transcription protein) peptide, VP22 (virus protein22) peptide, mph-1-btm(mouse transcription inhibition factor-1-biomolecule transduction mortif), Penetratin, Buforin II, Transportan, Ku70, Prion, pVEC and Pep-1.

15. The method for preparing a non-activated Wnt inhibition polypeptide (WIP) according to claim 13, wherein the purification step comprises: (a) binding the polypeptide to nickel-titanium beads; (b) washing the polypeptide-bound beads with urea solution; (c) and then eluting the polypeptide using imidazole and salt-containing buffer.

16. A method for preparing a non-activated Wnt inhibition polypeptide (WIP), the method comprising the steps of:
(a) expressing PTD-EED-WAD fusion polypeptide by culturing bacteria transformed with a recombinant vector according to claim 9;
(b) denaturing two-dimensional and three-dimensional structures of the polypeptide or changing the two-dimensional and three-dimensional structures of the polypeptide into a one-dimensional linear structure by recovering the cell pellets from the culture broth to add urea solution to the resulting cell pellets; and
(c) purifying the denatured PTD-EED-WAD fusion polypeptide (WIP).

17. The method for preparing a non-activated Wnt inhibition polypeptide (WIP) according to claim 16, wherein the PTD is selected from the group consisting of: TAT (transactivator of transcription) (SEQ ID NO: 3), AntHD (Drosophila homeoprotein atennapedia transcription protein) peptide (SEQ ID NO: 15), VP22 (virus protein22) peptide, mph-1-btm (mouse transcription inhibitory factor-1-biomolecule transduction mortif), Penetratin, Buforin II (SEQ ID NO: 20), Transportan (SEQ ID NO: 19), Ku70 (SEQ ID NO: 21), Prion, pVEC and Pep-1 (SEQ ID NO: 23).

18. The method for preparing a non-activated Wnt inhibition polypeptide (WIP) according to claim 16, wherein the purification step comprises: (a) binding the polypeptide to nickel-titanium beads; (b) washing the polypeptide-bound beads with urea solution; (c) and then eluting the polypeptide using imidazole and salt-containing buffer.

19. A pharmaceutical composition for inhibiting the growth and metastasis of cancer cells comprising the non-activated WIP according to claim 1 as an active ingredient and a pharmaceutical carrier.

20. A pharmaceutical composition for the treatment of immune diseases comprising the non-activated WIP according to claim 1 as an active ingredient and a pharmaceutical carrier.

21. The pharmaceutical composition for the treatment of immune diseases according to claim 20, wherein said immune disease is arthritis.

22. A pharmaceutical composition for the inhibition of pulmonary fibrosis comprising the non-activated WIP according to claim 1 as an active ingredient and a pharmaceutical carrier.
